(19) United States
(12) Patent Application Publication
(10) Pub. No.: US 2026/0224244 A1
(43) Pub. Date: Aug. 6, 2026

(54) TRIANGULATION APPARATUS, INSTRUMENT SET, AND METHOD FOR SURGERIES ON THE SPINAL COLUMN

(71) Applicant: Joimax GmbH, Karlsruhe (DE)

(72) Inventors: Wolfgang RIES, Linkenheim (DE); Michael RAPP, Löchgau (DE)

(73) Assignee: Joimax GmbH, Karlsruhe (DE)

(21) Appl. No.: 19/630,603

(22) Filed: Mar. 27, 2026

Related U.S. Application Data
(63) Continuation of application No. PCT/EP2024/076880, filed on Sep. 25, 2024.

(30) Foreign Application Priority Data
Sep. 28, 2023 (DE) 10 2023 126 542.3

(51) Int. Cl. A61B 17/34

(52) U.S. Cl. CPC A61B 17/3423

(57) ABSTRACT

A triangulation apparatus for at least two instruments for surgeries on the spinal column. The triangulation apparatus comprises at least two first access openings, through each of which the first instrument can be inserted, and at least one second access opening, through which the second instrument can be inserted. The two first access openings are arranged relative to the second access opening such that the trajectories through the two first access openings intersect the trajectory through the second access opening at different distances distally from the triangulation apparatus. The invention also relates to an instrument set having a first instrument, a second instrument, and the triangulation apparatus according to the invention, and to a method for spinal column surgery using the triangulation apparatus according to the invention.

This nonprovisional application is a continuation of International

Application No. PCT/EP2024/076880, which was filed on Sep. 25, 2024, and which claims priority to German Patent Application No. 10 2023 126 542.3, which was filed in Germany on Sep. 28, 2023, and which are both herein incorporated by reference.

### BACKGROUND OF THE INVENTION

### Field of the Invention

The invention relates to a triangulation apparatus for at least two instruments for surgeries on the spinal column, to an instrument set comprising a first instrument, with a second instrument and with the triangulation apparatus, and to a method of surgeries on the spinal column.

### Description of the Background Art

US 2022/0071645 A1 discloses a triangulation apparatus with which a bone drill can be guided at different angles toward the facet bone of a vertebral body in order to process the facet bone from several access directions as part of a surgical procedure on the spinal column. For this purpose, the known triangulation apparatus comprises three access openings having different angles of inclination in order to guide the bone drill toward the vertebral body at different angles. Another access opening of the triangulation apparatus is provided for a cannula to surround the nerve roots at the process of the vertebral body in order to protect nerve roots during processing of the vertebral body. The access openings of the triangulation apparatus are configured in such a manner that the bone drill always meets the cannula at the same distance distal to the triangulation apparatus, regardless of the access opening through which the bone drill is inserted. This fixed and unchangeable distance, at which the two instruments always meet distal to the triangulation apparatus, means that with a particular triangulation apparatus the facet bone to be processed must always be positioned at a fixed distance distally of the triangulation apparatus, and that if the distance deviates from this fixed distance, the triangulation apparatus can no longer be used, as otherwise there is a risk of serious tissue damage. In other words, a certain triangulation apparatus can only be used for patients having a certain body geometry, for example with a certain body mass index (BMI), but not for patients having different body geometries. The applicability of the known triangulation apparatus is therefore severely limited.

### SUMMARY OF THE INVENTION

It is therefore an object of the present invention to provide a triangulation apparatus with which the disadvantages of the prior art can be eliminated and by means of which, in particular, an extended range of applications is ensured.

The object of the invention is achieved by a triangulation apparatus for at least two instruments for surgeries on the spinal column, comprising at least two first access openings through which the first instrument can be inserted, with at least one second access opening through which the second instrument can be inserted, wherein the at least two first access openings are arranged relative to the at least one second access opening in such that the trajectories through the two first access openings intersect the trajectory through the second access opening at different distances distal to the triangulation apparatus.

The object is also achieved by an instrument set according an example of the invention for surgeries on the spinal column comprises a first instrument, a second instrument and a triangulation apparatus according to the invention, wherein the first instrument can be inserted through the at least two, in particular through all of the first access openings and wherein the second instrument can be inserted through the second access opening.

Furthermore, the object is achieved by a method that is a surgical procedure on the spinal column at a surgical site with a triangulation apparatus according to the invention, in particular with an instrument set according to the invention, having the following steps: creating an access to the surgical site, inserting the second instrument through the access to the surgical site, arranging the triangulation apparatus in such a manner that the second instrument passes through the second access opening of the triangulation apparatus, and inserting the first instrument distally through one of the first access openings of the triangulation apparatus in such a manner that the first instrument substantially comes into operative interaction, in particular into contact, with the second instrument.

A basic idea of the invention is to extend the applicability of a bipolar access to the surgical site in the context of spinal column surgical procedure with the aid of the triangulation apparatus according to the invention. This is made possible by the intersection points of the trajectories from the triangulation apparatus arranged at different distal distances, since, within the meaning of the invention the trajectories through the access openings correspond to the directions of extension of the instruments actually arranged therein, so that the intersection points of the trajectories correspond to those points at which the instruments inserted through the access openings meet. Direct mechanical contact between the instruments is not necessarily required; however, the distance between the instruments should be sufficiently small that the instruments are in an operative interaction with one another, for example if a working instrument as the first instrument at the surgical site can be visually inspected by an endoscope as the second instrument, wherein the first instrument is inserted through a first access opening and the second instrument is inserted through the second access opening.

In the context of the invention, such a surgical site can be, for example, the vertebral arch (arcus vertebrae) of a vertebral body or its facet joint. The surgical site can be located at different distances from the access site, i.e., the location where instruments are inserted into the patient's body as part of a surgical procedure. This variance in distances depends, among other things, on the patient's individual body geometry in general, for example on the patient's body mass index (BMI), but also on the specific vertebral body of the patient to be treated. By providing intersection points of the trajectories through the access openings, and thus also for the instruments inserted therein, at different distal distances, the triangulation apparatus according to the invention is suitable for a variety of anatomical conditions, in particular different patient body geometries.

The triangulation apparatus according to the invention provides intersection points for the instruments inserted into the access openings at various distances due to the intersection points of the trajectories, wherein the latter are also referred to as “kissing points” in order to illustrate the meeting of the instruments, in particular their operative interaction in these positions. The target point of an individual instrument at the surgical site is also referred to as the “landing point”. Accordingly, the triangulation apparatus according to the invention ensures that the target point of the first instrument coincides with the target point of the second instrument at the common intersection point. Within the meaning of the invention, a distal direction points towards the patient and a proximal direction towards the surgeon.

The first instrument associated with the at least two first access openings are associated may be a working instrument for the surgical procedure, which is configured for processing or guiding in human tissue. In this respect, the first instrument can be at least one member of the following group: needle, guide rod, guide tube, dilator, working sleeve, drill, milling cutter. The diameters of the first access openings can be configured to accommodate several first instruments in succession, such that the diameters of the first access openings each substantially correspond to the outer diameter of the largest first instrument to be inserted therein. A guide rod and/or a guide tube can serve as a guide for instruments to be inserted further toward the surgical site during the surgical procedure. A dilator expands human tissue as atraumatically as possible before the introduction of additional instruments, in particular, larger instruments. A working sleeve enables the insertion of additional working instruments, such as an endoscope or other instruments, which are inserted through the working sleeve to protect the surrounding tissue.

The outer diameters of the first access openings are in particular matched to the first instrument such that the first instrument can be inserted into the first access openings in a longitudinally displaceable manner, preferably with a tight fit.

The second instrument can be an inspection instrument, in particular an endoscope. Accordingly, the diameter of the second access opening can be matched to the diameter of the second instrument in such a manner that the second instrument can be inserted into the second access opening in a longitudinally displaceable manner, in particular with a tight fit.

Preferably, the at least two first access openings can each have a circular and/or oval cross-section. In particular, all of the first access openings have a circular and/or oval cross-section. A circular cross-section may be useful to allow the first instrument, which may have a circular cross-section, to be inserted into the triangulation apparatus with as little lateral play as possible and held in its current position relative to the triangulation apparatus. In particular when the first instrument is a working instrument within the meaning of the invention, the risk of tissue injuries due to unintentional movement of the working instrument is thereby reduced. In an advantageous further example of the invention, at least one first access opening can be designed as a bore and/or elongated hole. Preferably, all first access openings are configured as elongated holes. The longitudinal direction of the elongated hole of a first access opening can be arranged parallel to the direction of a connecting line between the first access opening and the second access opening. The elongated hole of the first access opening can have a length of between 1.5 mm and 20 mm, preferably about 12 mm, and/or a width of between 1.5 mm and 6.5 mm, preferably about 2.5 mm.

Preferably, the triangulation apparatus can comprise two, three, four or at least four first access openings. It may be provided that trajectories through two first access openings intersect the trajectory through the second access opening distal to the triangulation apparatus at different angles. In this manner, the first instrument can approach the “kissing point”, i.e., the intersection with the second instrument inserted through the second access opening, at different angles. This increases the processing possibilities at the surgical site by means of the first instrument, wherein it is ensured that the first instrument reaches the surgical site at the intended distal distance from the triangulation apparatus, regardless of the first access opening through which it is inserted. Incorrect positioning of the first instrument, especially along the proximal-distal direction, are thus avoided.

Preferably, at least one first access opening can be arranged with an angle of inclination other than 0° relative to the vertical. Within the meaning of the invention, the angle of inclination of an access opening corresponds to the angle that the trajectory through this access opening makes with respect to the vertical. In a further example of the invention, at least two, in particular all, of the first access openings are arranged with an angle of inclination other than 0° relative to the vertical. This ensures that the first instrument is guided to the surgical site at an angle that avoids, as far as possible, unintended tissue damage in the area of the spinal column, in particular to nerve structures. The triangulation apparatus according to the invention can therefore be used for procedures on the spinal column from a lateral direction.

Preferably, the angle of inclination of at least one first access opening relative to the vertical can be between 20° and 50°, in particular between 20° and 46°, preferably between 22° and 43°, in particular about 34°. This applies in particular to the angles of inclination of all the first access openings. All first access openings can have the same angle of inclination. In a further example of the invention, it may be provided that the angles of inclination of at least two first access openings, in particular of all first access openings, differ from one another.

Preferably, the angle of inclination of the first access opening facing the second access opening can be greater than the angle of inclination of the first access opening facing away from the second access opening. In particular, it may be provided that the angle of inclination of the first access opening facing the second access opening is the largest and/or that the angle of inclination of the first access opening facing away from the second access opening is the smallest. The triangulation apparatus may have, in the vicinity of a first access opening, information corresponding to the angle of inclination of the first access opening and/or the distance of the intersection point of the trajectory through the first access opening with the trajectory through the second access opening from the triangulation apparatus.

Preferably, the at least two first access openings, in particular all of the first access openings, can be arranged along a straight connecting line, wherein the connecting line is oriented in particular parallel to a directional component of the trajectory through one of the first access openings. The connecting line can run through the at least one second access opening, wherein the connecting line in particular runs through all first access openings and all second access openings and thus corresponds to a connecting line between the access openings.

At least three first access openings may be provided. The distance from a first first access opening to a second first access opening can be the same as the distance from the second first access opening to the third first access opening. All first access openings can be arranged in equal sizes, especially on the proximal end face and/or on the distal end face of the triangulation apparatus. In particular, in the case where all the first access openings are arranged with an angle of inclination other than 0° with respect to the vertical, they may be equally distributed on the proximal end face of the triangulation apparatus; however, this does not necessarily apply to the arrangement of the first access openings on the distal end face of the triangulation apparatus. The reverse also applies, i.e., an equally distributed arrangement of the first access openings on the distal end face does not necessarily imply an equally distributed arrangement on the proximal end face. The at least two first access openings, in particular all access openings, can be arranged in an area of the triangulation apparatus between its planar center and a side edge of the triangulation apparatus. In this respect, the triangulation apparatus may be spatially and functionally divided and comprise a first area associated with the first access openings and a second area associated with the second access opening, wherein the first area does not overlap with the second area. In a further example of the invention, the first access openings can be arranged in such a manner that the intersection points of the trajectories through the first access openings with the trajectory through the second access opening are each equidistant from one another, which applies in particular to their distal distances.

Preferably, only one second access opening can be provided. The second access opening can be configured to accommodate an inspection instrument, in particular an endoscope, as the second instrument. The cross-section of the second access opening may substantially correspond to the cross-section of the second instrument.

Preferably, the diameter of the second access opening can be larger than the diameter of at least one first access opening, in particular larger than the diameter of all first access openings. The cross-section of the second access opening can be larger than the cross-section of a first access opening, in particular larger than the cross-section of all first access openings.

Preferably, the second access opening can be configured as an elongated hole. In particular, if the second instrument has a circular cross-section, this allows fine adjustment of the second instrument inserted into the second access opening during procedures, wherein the second instrument is held in the second access opening but can be tilted about an axis, for example. The slight mobility of the second instrument made possible by the geometry of the second access opening provides an improved possibility for visual inspection of the surgical site when the second instrument is an inspection instrument. The second instrument can therefore still be moved slightly in the optimum viewing direction. The second access opening can have a length between 6.5 mm and 20 mm, preferably about 9.0 mm, and/or a width between 5 mm and 10 mm, preferably about 6.5 mm.

Preferably, the angle of inclination of the second access opening can be smaller than the angle of inclination of at least one first access opening, in particular smaller than the angle of inclination of all the first access openings. This ensures that, for example, an inspection instrument as the second instrument can reach the surgical site as directly as possible and inspect the first instrument there, while still minimizing tissue damage and trauma caused by the insertion of the second instrument, since anatomical conditions along the path to and at the surgical site are taken into account. For the first instrument, a larger angle of inclination relative to the second instrument is generally advantageous in order to guide the first instrument to the surgical site from a desired direction.

Preferably, the angle of inclination of the second access opening relative to the vertical can be other than 0° and is in particular between 10° and 40°, in particular between 10° and 20°, preferably substantially about 15°. This ensures that the second instrument can be inserted for visual inspection at the surgical site at an angle that is as safe as possible during surgeries on the spinal column.

Preferably, the intersection angle between the trajectory through the first access opening and the trajectory through the second access opening can be between 30° and 70°, in particular between 35° and 60°, preferably between 40° and 60° or between 45° and 55°, in particular approximately 49° or 50°. This applies in particular to all intersection angles between the trajectories through the first access openings and the trajectory through the second access opening. Taking into account the geometry of the patient at the surgical site, this enables both the first instrument and the second instrument to be inserted at a risk-free angle to the surgical site during surgeries on the spinal column, particularly in the case of an interlaminar, lateral, particularly posterolateral approach to the spinal canal of a patient, particularly taking into account the safety zone known as the “Kambin triangle” in the area of an intervertebral foramen. The intersection angle between the trajectory through the first access opening and the trajectory through the second access opening can be the sum of the inclination angles of the two trajectories relative to the vertical.

Preferably, the intersection points of the trajectories through the first access openings with the trajectory through the second access opening can be arranged distal to the triangulation apparatus at distances of from 15 mm to 100 mm, in particular from 15 mm to 25 mm or from 22.5 mm to 85 mm, preferably from 20 mm. This ensures that the intersection points of the trajectories are always located in the area in which the surgical site is spaced apart from the patient's skin surface on which the triangulation apparatus according to the invention can be placed as intended. The intersection points of the trajectories can be arranged axially aligned with one another, wherein a connecting line of the intersection points can be oriented normal to the end face of the triangulation apparatus. In a further example of the invention, it may be provided that the intersection points are laterally offset relative to one another, i.e., parallel to the end face of the triangulation apparatus, offset to one another.

Preferably, the triangulation apparatus can be movable between an open position and a closed position, wherein the access openings are at least partially open in the open position and closed in the closed position. The triangulation apparatus can be opened to insert and adjust the instruments and closed to secure the instruments in place. The triangulation apparatus can be opened and closed repeatedly.

Preferably, the triangulation apparatus can comprise at least two parts that are movably and/or captively connected to one another. The two parts can be pivotable relative to one another, in particular about a pivot axis, which can be oriented normal to the surface of the proximal end face of the triangulation apparatus and/or laterally offset relative to a connecting line of the access openings.

Preferably, the at least two first access openings and/or the at least one second access opening can be arranged along the contact surface of the two parts, in particular when the triangulation apparatus is in the closed position. This allows the instruments to be easily received in and secured within the access openings by corresponding movement of the two parts of the triangulation apparatus. In particular, all access openings are arranged along the contact surface of the two parts, which can thus correspond, at least in sections, to the connecting line of the access openings. The triangulation apparatus can be configured in such a manner that all instruments can be inserted into the access openings in a clamping manner. The contact surface of the two parts can, at least in sections, run parallel to a directional component of at least one trajectory through an access opening.

Preferably, the pivot axis of the parts of the triangulation apparatus that can pivot relative to one another can be arranged between the second access opening and the side wall of the triangulation apparatus facing the second access opening. This places the pivot axis close to the second instrument, which, as already mentioned, can be an endoscope. When the triangulation apparatus is opened by a pivoting movement of the two parts relative to one another and the access openings are thereby released, the access openings are not released to the same extent. The arrangement of the pivot axis in the vicinity of the second access opening ensures that the first access openings are largely released first, which allows readjustment of the first instruments, for example, while the second instrument remains comparatively firmly secured in the second access opening. The pivot axis can be arranged at a distance from the contact surface of the two parts of the triangulation apparatus. In particular, the pivot axis is arranged on the side of the triangulation apparatus associated with the second access opening, which may also be referred to as the endoscope side, thereby allowing easy opening of the opposite side associated with the first access openings, for example when using a scalpel as the first instrument to perform scalpel cuts. The pivot axis can be arranged spaced apart from the contact surface of the two parts of the triangulation apparatus. In a particular example of the invention, the pivot axis can be defined by a pin that is fixedly connected to one part of the triangulation apparatus and rotatably and, in particular, captively connected to the other part of the triangulation apparatus.

Preferably, the triangulation apparatus can be locked in the closed position, in particular clamped, in order to prevent unintentional misalignment of the instruments. The triangulation apparatus can be locked in the open position. For locking, the triangulation apparatus can have a clamp that can be configured as a clamping screw, which in particular passes through both parts of the triangulation apparatus. The clamping screw can have knurling on a lateral surface and/or a surface area of 300 mm² to 1000 mm², preferably 550 mm². The knurling makes the clamping screw easier to grip and operate. The clamping screw can have a diameter between 12 mm and 18 mm, preferably around 15 mm, and/or a height between 8 mm and 16 mm, in particular around 12 mm. A recess in the triangulation apparatus assigned to the clamping screw, through which the clamping screw passes in the closed position of the triangulation apparatus, can preferably be configured as a slot, such that the triangulation apparatus can be opened even when the clamping screw is only slightly loosened. The clamp can be arranged in an area of the triangulation apparatus between a first access opening, in particular the first access opening facing away from the second access opening, and a side edge of the triangulation apparatus facing this first access opening.

The triangulation apparatus can have an outwardly projecting, in particular convexly curved, protrusion on a side wall in order to simplify the handling of the triangulation apparatus, in particular its opening. The protrusion can be arranged on the side wall facing the first access opening. The protrusion can be arranged approximately at the height of the clamp such that the clamping screw can be easily loosened and the triangulation apparatus can subsequently be easily opened. Instead of the protrusion, or as an alternative thereto, the lateral outer wall can have a recess, in particular a concavely curved recess.

At least one first access opening and/or at least one second access opening can be associated with information on the triangulation apparatus that corresponds to the intersection point of the trajectory through the first access opening with the trajectory through the second access opening distal to the triangulation apparatus. The information corresponds in particular to the distal distance of the intersection point from the triangulation apparatus and/or its lateral position in relation to the proximal end face of the triangulation apparatus. In order to facilitate orientation of the triangulation apparatus and/or the instruments inserted therein, even during an X-ray inspection, the triangulation apparatus can have at least one radiographic marker.

The triangulation apparatus may have a length of between 50 mm and 180 mm, preferably 110 mm, and/or a width of between 25 mm and 75 mm, preferably 50 mm, and/or a height of between 5 mm and 20 mm, preferably 12 mm.

The first instrument can be inserted through all first access openings. The second instrument can be configured as an endoscope and can in particular have a working diameter of between 5 mm and 10 mm, in particular 6.5 mm or a maximum of 4 mm. The first instrument and/or the second instrument can have an atraumatic distal end face in order to avoid tissue injuries during the procedure.

The arrangement of the triangulation apparatus in the method according to the invention may provide that the triangulation apparatus, in the open position, is pushed from proximal to distal over the second instrument, for example an endoscope already arranged in the patient, wherein the second instrument is received in the second access opening and the triangulation apparatus is then moved into the closed position. Furthermore, it may be provided that access to the surgical site is initially created via the first access opening using a needle and a wire arranged therein, which is also known as the Seldinger technique. After insertion of the wire, the needle can be removed. The second instrument can be guided to the surgical site via the wire or without a previously placed needle or guide wire. The endoscope can have an obturator. During insertion of the first instrument, several dilators, in particular arranged coaxially and advanced one above the other, can be inserted as first instruments. The first instrument can be a guide rod, a guide sleeve, multiple guide sleeves, at least one dilator and/or a working sleeve. When inserting the first instrument through a first access opening of the triangulation apparatus, the first access opening can be selected depending on the desired angle and/or the desired distance of the intersection point with the second instrument already inserted through the second access opening. The first instrument can be moved independently of the second instrument. In particular, if the first instrument performs a tissue incision, this can be done efficiently and with relatively large instruments, particularly when the triangulation apparatus is open. The bipolar access to the surgical site provided by the invention allows the first instrument to be moved independently of the second instrument at the surgical site, resulting in improved flexibility in handling the instruments and thus improved performance of the procedure. Furthermore, compared to a monoportal access, an access with a reduced diameter is provided, since the instruments used for the procedure are not guided via a single access and the instruments do not have to be designed for combined use.

Further steps of the procedure can be performed after the first instrument has been inserted through the first access opening of the triangulation apparatus. The method may, for example, provide for at least one, in particular several dilators as first instruments to be advanced through the first access opening to the surgical site. It can then be provided that a working sleeve, as a further, in particular first, instrument, is advanced through the first access opening to the surgical site via the dilator already arranged at the surgical site. The at least one dilator, in particular all dilators located inside the working sleeve, can then be removed from the surgical site. In accordance with the invention, a working channel assigned to the first instrument is configured to be spatially separate from an inspection channel, which is assigned to the second instrument. After the procedure, all instruments and the triangulation apparatus according to the invention can be removed.

Further scope of applicability of the present invention will become apparent from the detailed description given hereinafter. However, it should be understood that the detailed description and specific examples, while indicating preferred embodiments of the invention, are given by way of illustration only, since various changes, combinations, and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1 is a schematic top view of a triangulation apparatus according to the invention;

FIG. 2 is a longitudinal section of the triangulation apparatus of FIG. 1 along the sectional line X-X in FIG. 1;

FIG. 3 is a bottom view of the triangulation apparatus of FIG. 1;

FIG. 4 is a cross-section through the triangulation apparatus of FIG. 3 along the sectional line B-B in FIG. 3;

FIG. 5 is a top view of a further example of the triangulation apparatus according to the invention;

FIG. 6 is a longitudinal section of the triangulation apparatus of FIG. 5 along the sectional line A-A in FIG. 5;

FIG. 7 is a bottom view of the triangulation apparatus of FIG. 5;

FIG. 8 is a cross-section through the triangulation apparatus of FIG. 7 along the sectional line B-B in FIG. 7; and

FIG. 9 is an instrument set according to the invention with a triangulation apparatus according to the invention during a surgical procedure on the spinal column and with a first instrument and with a second instrument, both of which pass through the triangulation apparatus.

### DETAILED DESCRIPTION

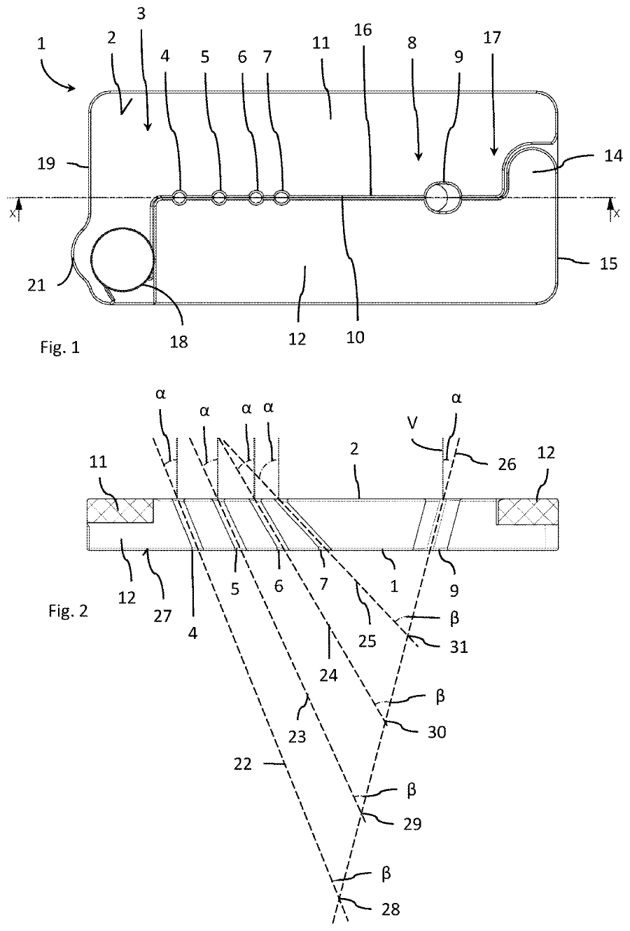

FIG. 1 is a triangulation apparatus 1 according to the invention for surgeries on the spinal column 36 in a schematic top view of its proximal end face 2. The triangulation apparatus 1 has a rectangular cross-section with rounded corners and has a length of 110 mm, a width of 50 mm and a height of 12 mm.

The proximal end face 2 of the triangulation apparatus 1 has four first access openings 4, 5, 6, 7, each having a circular cross-section, arranged in linear succession in a left-hand region of FIG. 1, each of which is adapted to a first instrument 34, in order to guide the instrument through the first access openings 4, 5, 6, 7 to a surgical site. As first instruments 35, working instruments for insertion into or processing of tissue can be used, for example needles, guide rods, guide tubes, dilators, working sleeves and/or bone drills or bone mills. The first access openings 4, 5, 6, 7 are arranged on the proximal end face 2 of the triangulation apparatus 1 at different distances from one another.

In a right-hand region 8 of the triangulation apparatus 1 in FIG. 1, a second access opening 9 is formed as an elongated hole. The second access opening 9 is arranged in alignment with the first access openings 4, 5, 6, 7 and is therefore located on a connecting line 10, which connects the first access openings 4, 5, 6, 7 with one another and which coincides with the sectional line X-X shown in FIG. 1. The depiction in FIG. 1 already shows that the second access opening 9 has an angle of inclination α relative to the vertical V other than 0°, which will be discussed later. The diameters of the first access openings 4, 5, 6, 7 are each smaller than the diameter of the second access opening 9; the same applies to the areas of the access openings 4, 5, 6, 7, 9. The second access opening 9 is designed for a second instrument 35, such as an inspection instrument, for example an endoscope, which can be inserted through the second access opening 9 and moved to the surgical site 37.

The triangulation apparatus 1 comprises two parts 11, 12, a first part 11 arranged at the top left in FIG. 1 and a second part 12 arranged at the bottom right in FIG. 1. The triangulation apparatus 1 can be moved between a closed position shown in FIG. 1 and an open position by pivoting the two parts 11, 12 relative to one another. The pivoting movement is effected by means of a pivot pin 13, which is concealed in FIG. 1 and which is fixed to the second part 12 and movably, but captively, connected to the first part 11. As a result, the pivoting movement takes place about a swivel axis 14 aligned normally to the proximal end face 2, the position of which is determined by the pin 13. In FIG. 1, the pivot axis 14 is offset upwards relative to the connecting line 10 of the access openings 4, 5, 6, 7, 9 and is arranged between the second access opening 9 and the side wall 15 of the triangulation apparatus 1 facing it. In the closed position of the triangulation apparatus 1, its parts 11, 12 are in contact with one another and form a contact surface 16, wherein all access openings 4, 5, 6, 7, 9 are arranged at least along a portion of the contact surface 16. As a result, instruments 34, 35 inserted in the access openings 4, 5, 6, 7, 9 are held in the closed position of the triangulation apparatus 1 without external force in a positively locked manner perpendicular to their longitudinal direction and in a frictionally locked manner along their longitudinal direction, so that inadvertent movement of the instruments 34, 35 and thus misalignment is prevented. When the triangulation apparatus 1 is moved from the closed position to the open position, the first access openings 4, 5, 6, 7, and thus the first instrument 34, are initially released due to the arrangement of the pivot axis 14 in an area 17 facing the second access opening 9, while the second instrument 35 is still comparatively fixed in the second access opening 9 and is only released when the triangulation apparatus 1 continues to move to the open position.

The triangulation apparatus 1 can be locked in the closed position shown in FIG. 1, so that unintentional opening of the triangulation apparatus 1 is prevented. For this purpose, a clamping screw is provided as a clamp 18, which is arranged in a lower left area of the triangulation apparatus 1 in FIG. 1. The clamping screw 18 is located below the connecting line 10 of the access openings 4, 5, 6, 7, 9 and in an area 3 between the first access opening 4 facing away from the second access opening 9 and the side wall 19 of the triangulation apparatus 1 facing the latter. For improved handling, the clamping screw 18 has knurling on its lateral surface 20, and extends through the first part 11 and the second part 12 of the triangulation apparatus 1, so that the first part 11 is frictionally connected to the second part 12 when the clamping screw 18 is actuated. In order to facilitate the opening of the triangulation apparatus 1 after manual actuation of the clamping screw 18, the area of the side wall 19 of the first part 11 facing the clamping screw 18 has a convexly curved protrusion 21, by means of which an operator can perform the opening pivoting movement simply, quickly and reliably during the procedure.

FIG. 2 shows the triangulation apparatus 1 of FIG. 1 in a longitudinal section along the sectional line X-X in FIG. 1, which, as already mentioned, corresponds to the connecting line 10 of the access openings 4, 5, 6, 7, 9. This depiction shows that all access openings 4, 5, 6, 7, 9 are each arranged at an angle of inclination α other than 0° relative to the vertical V. Accordingly, all trajectories 22, 23, 24, 25, 26 shown as dashed lines in FIG. 2 through the access openings 4, 5, 6, 7, 9 have an angle α other than 0° relative to the vertical V, which corresponds to the angle of inclination α. In the illustrated example, the first access openings 4, 5, 6, 7, coming from left to right in FIG. 2, have angles of inclination α relative to the vertical V of approximately 22°, 25°, 30°and 43°. The second access opening 9 has an angle of inclination α of around 15°, which is therefore smaller than all the angles of inclination α of the first access openings 4, 5, 6, 7. The angle of inclination α of the first access opening 7 facing the second access opening 9 is greater than the angle of inclination α of the other first access openings 4, 5, 6 and is also greater than the angle of inclination α of the second access opening 9. All trajectories 22, 23, 24, 25, 26 through the access openings 4, 5, 6, 7, 9 each have a directional component arranged parallel to the direction of the connecting line 10 of the access openings 4, 5, 6, 7, 9.

Due to the arrangement of the first access openings 4, 5, 6, 7 and their angle of inclination α, the first access openings 4, 5, 6, 7 emerge from the distal end face 27 of the triangulation apparatus 1 arranged at the bottom in FIG. 2 at equal distances from one another, whereas they are not arranged at equal distances from one another on the proximal end face 2 of the triangulation apparatus 1, as already mentioned.

The intersection points 28, 29, 30, 31 of the trajectories 22, 23, 24, 25 through the first access openings 4, 5, 6, 7 with the trajectory 26 through the second access opening 9 are located distally of the triangulation apparatus 1 at different distances therefrom and are offset from one another laterally, i.e., parallel to the end face 27 of the triangulation apparatus 1. The trajectory 25 through the first access opening 4 facing the second access opening 9 intersects the trajectory 26 through the second access opening 9 at a distal distance of 22.5 mm at an intersection angle β of approximately 58°. The trajectory 22 through the first access opening 4 facing away from the second access opening 9 intersects the trajectory 26 through the second access opening 9 at a distance of 85 mm at an intersection angle β of approximately 37°. Although the component of the triangulation apparatus 1 shown in FIG. 2 top right has the same hatching as the first part 11 arranged top left, it corresponds to the second part 12, as can also be seen in FIG. 1.

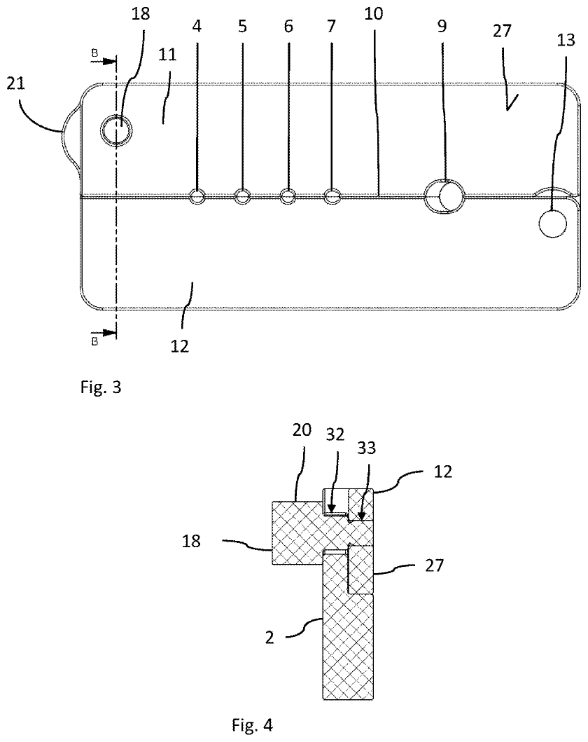

FIG. 3 shows the triangulation apparatus 1 in a view from below onto its distal end face 27. This also shows the arrangement of the first access openings 4, 5, 6, 7 at equal distances from one another as well as the inclined arrangement of the second access opening 9 relative to the vertical V. It is also shown that the clamping screw 18 extends through the two parts 11, 12 of the triangulation apparatus 1 and the pivot axis is configured by a pin 13 connected to the second part 12 and engaging in the first part 11. Both parts 11 and 12 are captively connected to each other.

FIG. 4 shows the triangulation apparatus 1 according to FIG. 3 in a cross-section along the sectional line B-B shown there, which is perpendicular to the connecting line 10 of the access openings 4, 5, 6, 7, 9 and is arranged at the height of the clamping screw 18. The clamping screw 18 engages through a recess 32 of the first part 11 configured as a slot and a recess 33 of the second part 12, wherein the latter is provided with a thread. The clamping screw 18 protrudes from the proximal end face 2 at a distance of 12 mm and is flush with the distal end face 27 of the triangulation apparatus 1 when it is screwed in and the triangulation apparatus 1 is locked in place.

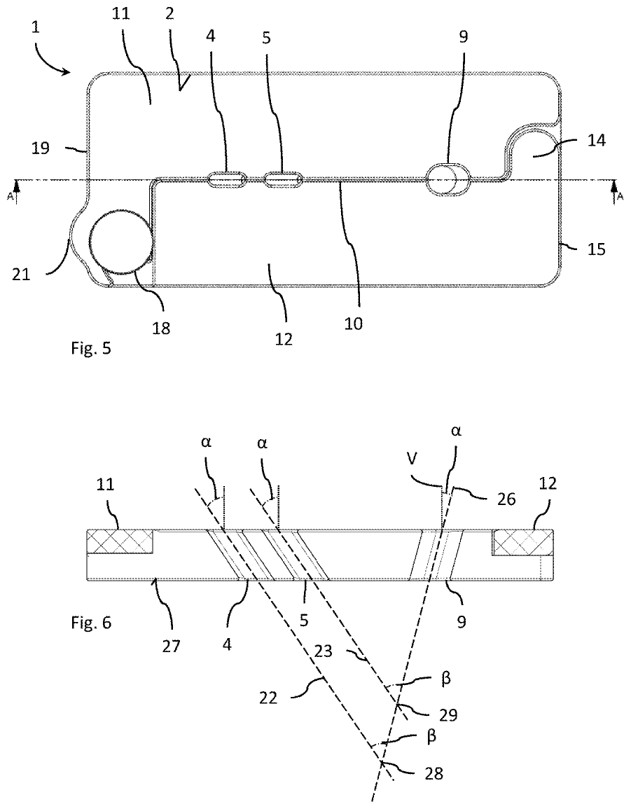

FIG. 5 shows a further example of the triangulation apparatus 1 according to the invention in a top view of its proximal end face 2. In contrast to the example shown in FIG. 1, the example shown here has two first access openings 4, 5 arranged one behind the other, which are configured as elongated holes. The elongated holes 4, 5 are oriented parallel to the connecting line 10 of the access openings. The width of the elongated holes 4, 5 corresponds approximately to the diameter of the first access openings 4, 5, 6, 7 as shown in FIG. 1. Further features of the triangulation apparatus 1 according to FIG. 1 are also present in the example according to FIG. 5, in particular the second access opening 9, the clamping screw 18 and the pivot axis 14.

FIG. 6 shows a section through the triangulation apparatus 1 according to FIG. 5 along the sectional line A-A shown there, which corresponds to the connecting line 10 of the access openings 4, 5, 9. It can be seen from this that the first access openings 4, 5 each have the same angle of inclination α relative to the vertical V, which in the illustrated example is approximately 34°. The distance between the two first access openings 4, 5 on the proximal end face 2 of the triangulation apparatus 2 therefore corresponds to their distance on the distal end face 27 of the triangulation apparatus 1. The inclination angles α of the first access openings 4, 5 are each greater than the inclination angle α of the second access opening 9, which incidentally corresponds to the inclination angle α of the second access opening 9 of the example according to FIG. 1 and is approximately 15°. Since both first access openings 4, 5 each have the same angle of inclination α, the trajectories 22, 23 through the first access openings 4, 5 each intersect the trajectory 26 through the second access opening 9 at the same intersection angle β, in this case approximately 49°. The two intersection points 28, 29 are arranged at different distal distances from the triangulation apparatus 1 and laterally offset from one another.

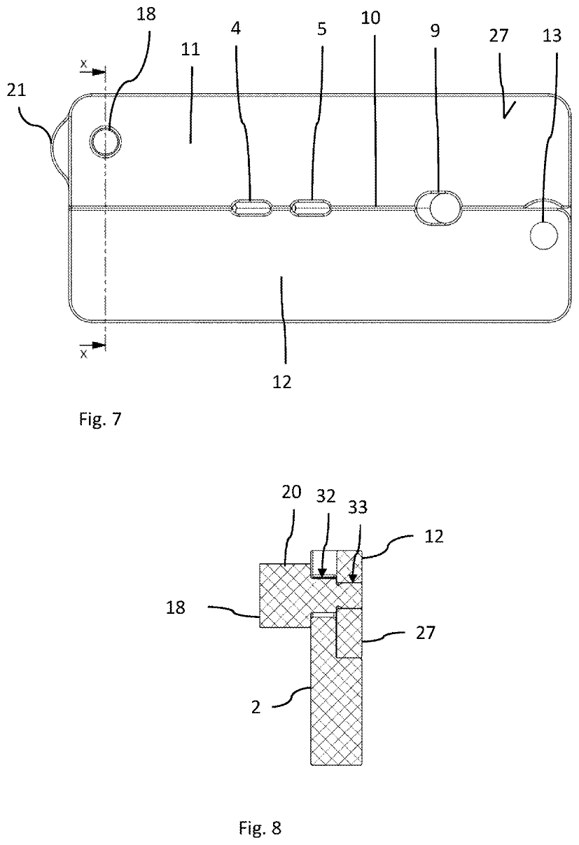

FIG. 7 shows the triangulation apparatus 1 according to FIG. 5 in a view from below of its distal end face 27 and thus in particular the positions of the access openings 4, 5, 9 emerging therefrom as well as the clamping screw 18 and the pin 13.

FIG. 8 shows a cross-section through the triangulation apparatus according to FIG. 7 along the sectional line X-X drawn there, which corresponds to the sectional line B-B according to FIG. 3. Therefore, this representation corresponds to FIG. 4, so reference is made to the above explanations to avoid repetition.

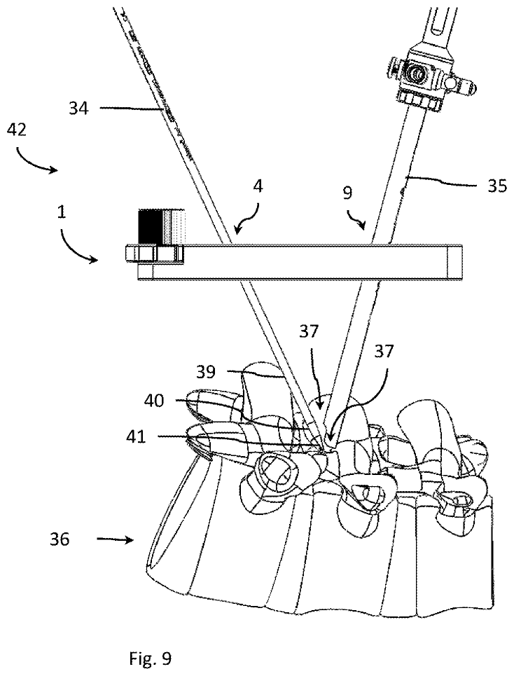

FIG. 9 shows an instrument set 42 according to the invention with a triangulation apparatus 1 according to the invention as part of a surgical procedure on the spinal column 36 of a patient, which is arranged at the bottom in FIG. 9. The triangulation apparatus 1 is in the closed position. The triangulation apparatus 1 rests on the skin surface at a predefined position such that a first instrument 34, arranged on the left in FIG. 9 and configured as a working instrument, passes through the first access opening 4 of the triangulation apparatus 1, which is perspectively covered in FIG. 9, and extends to the surgical site 37. In addition, a second instrument 35, configured as an endoscope in the example shown, passes through the second access opening 9 of the triangulation apparatus 1, which is also covered in FIG. 9. Thus, the first instrument 34 is oriented at the angle of inclination with respect to the vertical that corresponds to the angle of inclination of the first access opening 4. The same applies to the second instrument 35, whose angle of inclination corresponds to the angle of inclination of the second access opening 9. For reasons of clarity, the inclination angles and the trajectories of the access openings are not shown in FIG. 9.

The triangulation apparatus 1 is configured such that the intersection point 28 of the trajectory through the first access opening 4 with the trajectory through the second access opening 9 corresponds to the intersection point 37 of the first instrument 34 with the second instrument 35, wherein the intersection point 37 corresponds to the surgical site 37, is axially spaced from the triangulation apparatus 1 by a predefined value and results substantially from the arrangement and orientation of the access openings 4, 9. At the intersection point 37, tissue 38 is treated by means of the first instrument 34, which arranged radially one above the other-has a working sleeve 39, a dilator 40 for dilating the tissue 38 of the patient and a radially inner bone drill 41. At the same time, a visual inspection of the surgical site 37 is carried out using the endoscope as the second instrument 35. The instrument set 42 according to the invention comprises the triangulation apparatus 1 according to the invention, the first instrument 34, including its individual components 39, 40, 41, and the second instrument 35, including its individual components, if any.

Since the first instrument 34 and the second instrument 35 are in this respect in an operative interaction at the surgical site 37, this site is also referred to as the “kissing point” due to the proximity of the two instruments 34, 35.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. A triangulation apparatus for at least two instruments for surgeries on the spinal column, the triangulation apparatus comprising:
at least two first access openings through which the first instrument is adapted to be inserted;
at least one second access opening through which the second instrument is adapted to be inserted,
wherein the at least two first access openings are arranged relative to the at least one second access opening such that trajectories through the two first access openings intersect the trajectory through the second access opening at different distances distal to the triangulation apparatus.

2. The triangulation apparatus according to claim 1, wherein the at least two first access openings each have a circular and/or an oval cross-section.

3. The triangulation apparatus according to claim 1, wherein two, three, four or at least four first access openings are provided.

4. The triangulation apparatus according to claim 1, wherein at least one first access opening is arranged with an inclination angle other than 0° relative to a vertical.

5. The triangulation apparatus according to claim 4, wherein an angle of inclination of at least one first access opening with respect to the vertical is between 20° and 46°, or between 22° and 43°, or is 34°.

6. The triangulation apparatus according to claim 1, wherein the angle of inclination of the first access opening facing the second access opening is greater than the angle of inclination of the first access opening facing away from the second access opening.

7. The triangulation apparatus according to claim 1, wherein the at least two first access openings or all of the first access openings are arranged along a straight connecting line, and wherein the connecting line is oriented substantially parallel to a directional component of the trajectory through the respective first access opening.

8. The triangulation apparatus according to claim 1, wherein only one second access opening is provided.

9. The triangulation apparatus according to claim 1, wherein the diameter of the second access opening is larger than the diameter of at least one first access opening or is larger than the diameters of all the first access openings.

10. The triangulation apparatus according to claim 1, wherein the second access opening is formed as a bore and/or elongated hole.

11. The triangulation apparatus according to claim 1, wherein the angle of inclination of the second access opening is smaller than the angle of inclination of one of the first access openings or is smaller than the angles of inclination of all the first access openings.

12. The triangulation apparatus according to claim 1, wherein the angle of inclination of the second access opening with respect to the vertical is other than 0° and or is between 10° and 20°, or is 15°.

13. The triangulation apparatus according to claim 1, wherein an intersection angle between a trajectory through the first access opening and a trajectory through the second access opening is between 40° and 60°, or between 45° and 55°, or is about 49°.

14. The triangulation apparatus according to claim 1, wherein the intersection points of the trajectories through the first access openings with the trajectory through the second access opening are arranged distal to the triangulation apparatus at distances of from 15 mm to 100 mm, or from 22.5 mm to 85 mm.

15. The triangulation apparatus according to claim 1, wherein the triangulation apparatus is movable between an open position and into a closed position, and wherein the access openings are at least partially released in the open position and closed in the closed position.

16. The triangulation apparatus according to claim 1, wherein the triangulation apparatus has at least two parts that are movably and/or captively connected to one another relative to one another, or are pivotable.

17. The triangulation apparatus according to claim 16, wherein the at least two first access openings and/or the at least one second access opening are arranged along the contact surface of the two parts.

18. The triangulation apparatus according to claim 16, wherein the pivot axis of the parts of the triangulation apparatus that can pivot relative to one another is arranged between the second access opening and the side edge of the triangulation apparatus facing the second access opening.

19. The triangulation apparatus according to claim 15, wherein the triangulation apparatus is adapted to be locked or clamped in the closed position.

20. An instrument set for surgeries on the spinal column comprising:
a first instrument;
a second instrument; and
the triangulation apparatus according to claim 1,
wherein the first instrument is insertable through at least one or through all first access openings, and
wherein the second instrument is insertable through the second access opening.

21. A method for surgeries on the spinal column at a surgical site, the method comprising:
providing the triangulation apparatus according to claim 1;
creating an access to the surgical site;
inserting the second instrument through the access to the surgical site;
arranging the triangulation apparatus such that the inserted second instrument passes through the second access opening of the triangulation apparatus; and
inserting the first instrument distally through one of the first access openings of the triangulation apparatus such that the first instrument comes into operative interaction with the second instrument.
